(19) United States
(12) Patent Application Publication
(10) Pub. No.: US 2016/0120865 A1
(43) Pub. Date: May 5, 2016

(54) TOPICAL TREATMENT OF LOCALIZED SCLERODERMA

(71) Applicant: PRECISION DERMATOLOGY, INC., Cumberland, RI (US)

(72) Inventors: Mark W. Trumbore, Westford, MA (US); Martin P. Redmon, Oxford, MA (US); Robert Alan Lafyatis, Arlington, MA (US); Pinaki Ranjan Majhi, Sharon, MA (US)

(21) Appl. No.: 14/897,516

(22) PCT Filed: May 30, 2014
(86) PCT No.: PCT/US14/40189

§ 371 Date: Dec. 10, 2015

Related U.S. Application Data
(60) Provisional application No. 61/844,983, filed on Jul. 11, 2013.

(51) Int. Cl. A61K 31/506; A61K 9/06; A61K 9/00

(52) U.S. Cl. CPC A61K 31/506; A61K 9/0014; A61K 9/06

(57) ABSTRACT

Disclosed are compositions and formulations for topical administration that contain a tyrosin kinase inhibitor, such as imatinib or nilotinib. The topical compositions or formulations are useful in treating scleroderma.

### RELATED APPLICATIONS

This application claims the benefit of priority to U.S. Provisional Patent Application Ser. No. 61/844,983, filed Jul. 11, 2013, which is hereby incorporated by reference.

### BACKGROUND

Systemic sclerosis, also known as scleroderma, is a chronic systemic autoimmune disease (primarily of the skin) characterized by fibrosis (or hardening), vascular alterations, and autoantibodies. There are two major forms: (1) limited systemic sclerosis/scleroderma involves cutaneous manifestations that mainly affect the hands, distal arms, and face. It was previously called CREST syndrome in reference to the following complications: Calcinosis, Raynaud's phenomenon, Esophageal dysfunction, Sclerodactyly, and Telangiectasias. Additionally, pulmonary arterial hypertension may occur in up to one-third of patients and is the most serious complication for this form of scleroderma; and (2) diffuse systemic sclerosis/scleroderma is rapidly progressing and affects more proximal skin and one or more internal organs, frequently the kidneys, esophagus, heart, and lungs. Both forms of scleroderma can be disabling and life-threatening.

There are currently no treatments for scleroderma itself, but individual organ system complications may be treated, and specific symptoms ameliorated. Although fibrosis of the skin has been treated with various agents, such as D-penicillamine, mycophenolate, colchicine, psoralen+ultraviolet A exposure (PUVA), relaxin, cyclosporine, and EPA (omega-3 oil derivative), none of them has been proven to be beneficial in a controlled trial. Also, there are currently no medications or treatments for scleroderma skin disease approved by the U.S. Food and Drug Administration (FDA).

Tyrosine kinases are enzymes responsible for the activation of many proteins by signal transduction cascades. Tyrosine kinase inhibitors (TKIs) operate by four different mechanisms: they can compete with adenosine triphosphate (ATP), the substrate, or both, or can act in an allosteric fashion, namely binding to a site outside the active site of the enzyme, affecting its activity by a conformational change. Oral or intraperitoneal (IP) administration of TKIs, such as imatinib and nilotinib, have been reported to be effective in reducing clinical signs associated with scleroderma in mice. However, systemic use of these molecules is associated with significant adverse effects, and to date clinical trials in patients with scleroderma have not shown any consistent benefit associated with these agents.

In most cells transforming growth factor beta (TGF-β) is a protein that controls proliferation, cellular differentiation, and other functions. It is a type of cytokine which plays a role in immunity, cancer, bronchial asthma, heart disease, diabetes, Marfan syndrome, Loeys-Dietz syndrome, Parkinson's disease, and AIDS. TGF-β acts as an antiproliferative factor in normal epithelial cells and is a potent stimulator of fibrosis both in vitro and in vivo. TGF-β has been strongly implicated as the cytokine mediating fibrosis in scleroderma. Examples of inhibitors of TGF-β include, but are not limited to, SB-431542, SD-208, A 83-01, D 4476, GW 788388, RepSox, SB 505124, and SB 525334.

There exists a need for an effective treatment for cutaneous scleroderma.

### SUMMARY OF THE INVENTION

In certain embodiments, the invention relates to a method of treating scleroderma, comprising the step of applying topically to an affected area of the skin of a subject in need thereof a composition or a formulation comprising a therapeutically effective amount of a tyrosine kinase inhibitor, and a dermatologically acceptable carrier or excipient.

In certain embodiments, the invention relates to any one of the aforementioned methods, wherein the tyrosine kinase inhibitor is effective against BCR-ABL tyrosine kinase, c-Abl tyrosine kinase, α-PDGFR, β-PDGFR, or KIT receptor kinase, or inhibits TGF-β signaling through these or other tyrosine kinases.

In certain embodiments, the invention relates to any one of the aforementioned methods, wherein the tyrosine kinase inhibitor is imatinib or nilotinib.

In certain embodiments, the invention relates to a composition or a formulation, comprising a therapeutically effective amount of a tyrosine kinase inhibitor, and a dermatologically acceptable carrier or excipient.

### BRIEF DESCRIPTION OF THE FIGURES

FIG. 1 depicts the release profile of imatinib mesylate through each of two synthetic membranes.

FIG. 2 is a drawing of a mouse with labels for areas of skin treated with various compositions (LS=local (i.e., treated) skin; +DS=near (i.e., proximal) local skin; −DS=far (i.e., distal) local skin).

FIGS. 3-13 depict graphically the relative magnitudes of expression of various genes in each of two areas of skin of mice treated topically with various compositions comprising imatinib or without imatinib (control).

### DETAILED DESCRIPTION OF THE INVENTION

### Overview

In certain embodiments, the invention relates to a topical formulation comprising a therapeutically effective amount of a tyrosine kinase inhibitor or a TGF-β inhibitor. In certain embodiments, topical application of these agents applies the drug directly to the affected tissue, greatly reducing systemic exposure and adverse effects while maximizing therapeutic efficacy. In certain embodiments, the inhibitor is chemically stable in the formulation. In certain embodiments, the inhibitor is readily released from the formulation. In certain embodiments, the inhibitor is able to penetrate into the skin once applied. In certain embodiments, the formulation can be in the form of a cream, lotion, solution, gel, ointment, spray, aerosol spray, or aerosol foam.

In certain embodiments, the topical formulations of the invention are non-irritating in vitro and in vivo. In certain embodiments, the topical formulations are well-tolerated when applied daily for periods of one to two months.

In certain embodiments, the topical formulations of the invention are effective in treating localized scleroderma as demonstrated by their ability to (i) generate responses in disease-relevant biomarkers, (ii) improve modified local scleroderma skin severity index scores, or (iii) improve a physician's global assessment of disease activity while demonstrating lower systemic bioavailability compared to oral or parenteral dosing.

In certain embodiments, the topical formulations of the invention are applied one to three times daily (or more) to disease-affected areas of patients. Affected patients can be in either active (inflammation, sclerosis) or damaging (atrophic) disease stages, and can have either progressive disease or disease in remission. Patients may be chosen based upon their exhibiting molecular markers consistent with disease drivers as determined by pre-treatment immunohistochemistry (IHC) and/or microarray assays.

### DEFINITIONS

For convenience, certain terms employed in the specification and appended claims are collected here. These definitions should be read in light of the entire disclosure and understood as by a person of skill in the art.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The phrase “or,” as used herein in the specification and in the claims, should be understood to mean “either or both” of the elements so conjoined, i.e., elements that are conjunctively present in some cases and disjunctively present in other cases. Multiple elements listed with “or” should be construed in the same fashion, i.e., “one or more” of the elements so conjoined. Other elements may optionally be present other than the elements specifically identified by the “or” clause, whether related or unrelated to those elements specifically identified. Thus, as a non-limiting example, a reference to “A or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### Exemplary Constituents of Compositions of the Invention

Listed below are exemplary identities of various constituents of the compositions of the present invention.

### 1. Propellants

In certain embodiments, the propellant is a HFA or a mixture of one or more hydrofluorocarbons. Suitable hydrofluorocarbons include 1,1,1,2-tetrafluoroethane (HFA 134a); 1,1,1,2,3,3,3-heptafluoropropane (HFA 227); and mixtures and admixtures of these and other HFAs that are currently approved or may become approved for medical use are suitable. The concentration of the HFA propellant is from about 2% to about 50% by weight of the composition. In certain embodiments, the propellant comprises a hydrofluoroolefin (HFO), or a mixture of HFO and HFA. Suitable hydrofluoroolefins include 1,3,3,3-tetrafluoropropene (HFO 1234ze) and mixtures and admixtures of this and other HFO suitable for topical use. The concentration of the HFO propellant is from about 2% to about 50% by weight of the composition. Hydrocarbon as well as CFC propellants can also be used in the present invention.

### 2. Vehicles

Suitable topical vehicles and vehicle components for use with the formulations of the invention are well known in the cosmetic and pharmaceutical arts, and include such vehicles (or vehicle components) as water; organic solvents such as alcohols (particularly lower alcohols readily capable of evaporating from the skin such as ethanol), glycols (such as propylene glycol, butylene glycol, and glycerol (glycerin)), aliphatic alcohols (such as lanolin); mixtures of water and organic solvents (such as water and alcohol), and mixtures of organic solvents such as alcohol and glycerol (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, dimethiconol, dimethicone, and dimethicone copolyol; hydrocarbon-based materials such as petrolatum and squalane; and other vehicles and vehicle components that are suitable for administration to the skin, as well as mixtures of topical vehicle components as identified above or otherwise known to the art.

In one embodiment, the compositions of the present invention are oil-in-water emulsions. Liquids suitable for use in formulating compositions of the present invention include water, and water-miscible solvents such as glycols (e.g., ethylene glycol, butylene glycol, isoprene glycol, propylene glycol), glycerol, liquid polyols, dimethyl sulfoxide, and isopropyl alcohol. One or more aqueous vehicles may be present.

In one embodiment, formulations without methanol, ethanol, propanols, or butanols are desirable.

In one embodiment, the compositions of the invention are hydrophilic gels. Liquids suitable for use in formulating compositions of the invention include water, and water-miscible solvents, such as lower alcohols, glycols (e.g., ethylene glycol, butylene glycol, isoprene glycol, propylene glycol), glycerol, liquid polyols, dimethyl sulfoxide, and isopropyl alcohol. One or more aqueous or water-miscible vehicles may be present.

### 3. Surfactants and Emulsifiers

Many topical formulations contain chemical emulsions that use surface active ingredients (emulsifiers and surfactants) to disperse dissimilar chemicals in a particular solvent system. For example, most lipid-like (oily or fatty) or lipophilic ingredients do not uniformly disperse in aqueous solvents unless they are first combined with emulsifiers, which form microscopic aqueous soluble structures (droplets) that contain a lipophilic interior and a hydrophilic exterior, resulting in an oil-in-water emulsion. In order to be soluble in aqueous media, a molecule must be polar or charged so as to favorably interact with water molecules, which are also polar. Similarly, to dissolve an aqueous-soluble polar or charged ingredient in a largely lipid or oil-based solvent, an emulsifier is typically used which forms stable structures that contain the hydrophilic components in the interior of the structure while the exterior is lipophilic so that it can dissolve in the lipophilic solvent to form a water-in-oil emulsion. It is well known that such emulsions can be destabilized by the addition of salts or other charged ingredients which can interact with the polar or charged portions of the emulsifier within an emulsion droplet. Emulsion destabilization results in the aqueous and lipophilic ingredients separating into two layers, potentially destroying the commercial value of a topical product.

Surfactants suitable for use in the present invention may be ionic or non-ionic. These include, but are not limited to: cetyl alcohol, polysorbates (Polysorbate 20, Polysorbate 40, Polysorbate 60, Polysorbate 80), steareth-10 (Brij 76), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol, N,N-dimethyldodecylamine-N-oxide, hexadecyltrimethylammonium bromide (HTAB), polyoxyl 10 lauryl ether, bile salts (such as sodium deoxycholate or sodium cholate), polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, dicetyl phosphate (dihexadecyl phosphate), ceteareth-10 phosphate, methylbenzethonium chloride, dicetyl phosphate, ceteth-10 phosphate (ceteth-10 is the polyethylene glycol ether of cetyl alcohol where n has an average value of 10; ceteth-10 phosphate is a mixture of phosphoric acid esters of ceteth-10), ceteth-20, Brij S10 (polyethylene glycol octadecyl ether, average Mₙ˜711), and Poloxamers (including, but not limited to, Poloxamer 188 (HO(C₂H₄O)ₐ(CH(CH₃)CH₂O)_{b}(C₂H₄O)ₐH, average molecular weight 8400) and Poloxamer 407 (HO(C₂H₄O)ₐ(CH(CH₃)CH₂O)_{b}(C₂H₄O)ₐH, wherein a is about 101, and b is about 56)). Appropriate combinations or mixtures of such surfactants may also be used according to the present invention.

Many of these surfactants may also serve as emulsifiers in formulations of the present invention.

Other suitable emulsifiers for use in the formulations of the present invention include, but are not limited to, behentrimonium methosulfate-cetearyl alcohol, non-ionic emulsifiers like emulsifying wax, polyoxyethylene oleyl ether, PEG-40 stearate, cetostearyl alcohol (cetearyl alcohol), ceteareth-12, ceteareth-20, ceteareth-30, ceteareth alcohol, Ceteth-20 (Ceteth-20 is the polyethylene glycol ether of cetyl alcohol where n has an average value of 20), oleic acid, oleyl alcohol, glyceryl stearate, PEG-75 stearate, PEG-100 stearate, and PEG-100 stearate, ceramide 2, ceramide 3, stearic acid, cholesterol, steareth-2, and steareth-20, or combinations/mixtures thereof, as well as cationic emulsifiers like stearamidopropyl dimethylamine and behentrimonium methosulfate, or combinations/mixtures thereof.

### 4. Moisturizers, Emollients, and Humectants

One of the most important aspects of a topical product is consumers' perceptions of the aesthetic qualities of the product. For example, while white petrolatum is an excellent moisturizer and skin protectant, it is rarely used alone, especially on the face, because it is greasy, sticky, does not rub easily into the skin and may soil clothing. Consumers highly value products which are aesthetically elegant and have an acceptable tactile feel and performance on their skin.

Suitable moisturizers for use in the formulations of the present invention include, but are not limited to, lactic acid and other hydroxy acids and their salts, glycerol, propylene glycol, butylene glycol, sodium PCA, sodium hyaluronate, Carbowax 200, Carbowax 400, and Carbowax 800.

Suitable emollients or humectants for use in the formulations of the present invention include, but are not limited to, panthenol, cetyl palmitate, glycerol (glycerin), PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, 2-ethylhexyl palmitate (octyl palmitate), dimethicone, phenyl trimethicone, cyclomethicone, C₁₂-C₁₅ alkyl benzoates, dimethiconol, propylene glycol, Theobroma grandiflorum seed butter, ceramides (e.g., ceramide 2 or ceramide 3), hydroxypropyl bispalmitamide MEA, hydroxypropyl bislauramide MEA, hydroxypropyl bisisostearamide MEA, 1,3-bis(N-2-(hydroxyethyl)stearoylamino)-2-hydroxy propane, bis-hydroxyethyl tocopherylsuccinoylamido hydroxypropane, urea, aloe, allantoin, glycyrrhetinic acid, safflower oil, oleyl alcohol, oleic acid, stearic acid, dicaprylate/dicaprate, diethyl sebacate, isostearyl alcohol, pentylene glycol, isononyl isononanoate, and 1,3-bis(N-2-(hydroxyethyl)palmitoylamino)-2-hydroxypropane.

In addition, appropriate combinations and mixtures of any of these moisturizing agents and emollients may be used in accordance with the present invention.

### 5. Preservatives and Antioxidants

The composition may further include components adapted to improve the stability or effectiveness of the applied formulation.

Suitable preservatives for use in the present invention include, but are not limited to: ureas, such as imidazolidinyl urea and diazolidinyl urea; phenoxyethanol; sodium methyl paraben, methylparaben, ethylparaben, and propylparaben; potassium sorbate; sodium benzoate; sorbic acid; benzoic acid; formaldehyde; citric acid; sodium citrate; chlorine dioxide; quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; piroctone olamine; Vitis vinifera seed oil; and alcoholic agents, for example, chlorobutanol, dichlorobenzyl alcohol, phenylethyl alcohol, and benzyl alcohol.

Suitable antioxidants include, but are not limited to, ascorbic acid and its esters, sodium bisulfite, butylated hydroxytoluene, butylated hydroxyanisole, tocopherols (such as α-tocopherol), tocopheryl acetate, sodium ascorbate/ascorbic acid, ascorbyl palmitate, propyl gallate, and chelating agents like EDTA (e.g., disodium EDTA), citric acid, and sodium citrate.

In certain embodiments, antioxidants or preservatives of the present invention may also function as a moisturizer or emollient, for example.

In addition, combinations or mixtures of these preservatives or anti-oxidants may also be used in the formulations of the present invention.

### 6. Additional Active Agents

In addition to tyrosine kinase inhibitors, other active agents may be included in the formulation. The additional active agent may be any material that has a desired effect when applied topically to a mammal, particularly a human. Suitable classes of active agents include, but are not limited to, antibiotic agents, antimicrobial agents, anti-acne agents, antibacterial agents, antifungal agents, antiviral agents, steroidal anti-inflammatory agents, non-steroidal anti-inflammatory agents, anesthetic agents, antipruriginous agents, antiprotozoal agents, anti-oxidants, antihistamines, vitamins, and hormones. Steroidal and non-steroidal anti-inflammatory and keratolytic agents are especially suitable for use in combination with the topical tyrosine kinase inhibitors. Mixtures of any of these active agents may also be employed. Additionally, dermatologically-acceptable salts and esters of any of these agents may be employed.

6.1 Non-Steroidal Anti-Inflammatory Agents

Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac, fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone; and niacinamide. Mixtures of these non-steroidal anti-inflammatory agents may also be employed, as well as the dermatologically acceptable salts and esters of these agents. For example, etofenamiate, a flufenamic acid derivative, is particularly useful for topical application.

6.2 Steroidal Anti-Inflammatory Agents

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters (including betamethasone dipropionate), chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

6.3 Keratolytic Agents

Suitable keratolytic agents include, but are not limited to, urea, salicylic acid, papain, sulfur, glycolic acid, pyruvic acid, resorcinol, N-acetylcysteine, retinoids such as retinoic acid (e.g., tretinoin) and its derivatives (e.g., cis and trans isomers, esters), retinol, alpha hydroxy acids, beta hydroxy acids, coal tar, and combinations thereof.

### 7. Purging Gases

In one embodiment, the air in the container charged with the composition is replaced by an inert gas. In certain embodiments, the inert gas is selected from the group consisting of argon, nitrogen, and mixtures thereof

### 8. Buffer Salts

Suitable buffer salts are well-known in the art. Examples of suitable buffer salts include, but are not limited to acetate salts (e.g., sodium acetate), sodium citrate, citric acid, sodium phosphate monobasic, sodium phosphate dibasic, sodium phosphate tribasic, potassium phosphate monobasic, potassium phosphate dibasic, and potassium phosphate tribasic.

### 9. Viscosity Modifiers and Gelants

Suitable viscosity adjusting agents (i.e., thickening and thinning agents or viscosity modifying agents) for use in the formulations of the present invention include, but are not limited to, protective colloids or non-ionic gums such as carboxymethylcellulose, hydroxyethylcellulose, xanthan gum, and sclerotium gum, as well as magnesium aluminum silicate, sodium magnesium fluorosilicate, silica, microcrystalline wax, beeswax, paraffin, and cetyl palmitate. In addition, appropriate combinations or mixtures of these viscosity adjusters may be utilized according to the present invention.

### 10. Additional Constituents

Additional constituents suitable for incorporation into the formulations of the invention include, but are not limited to: skin protectants, adsorbents, demulcents, emollients, moisturizers, sustained release materials, solubilizing agents, skin-penetration agents, skin soothing agents, deodorant agents, antiperspirants, sun screening agents, sunless tanning agents, vitamins, hair conditioning agents, anti-irritants, anti-aging agents, abrasives, absorbents, anti-caking agents, anti-static agents, astringents (e.g., witch hazel, alcohol, and herbal extracts such as chamomile extract), binders/excipients, buffering agents, chelating agents, film forming agents, conditioning agents, opacifying agents, lipids, immunomodulators, and pH adjusters (e.g., citric acid, sodium hydroxide, and sodium phosphate).

For example, lipids normally found in healthy skin (or their functional equivalents) may be incorporated into the emulsions of the present invention. In certain embodiments, the lipid is selected from the group consisting of ceramides, cholesterol, and free fatty acids. Examples of lipids include, but are not limited to, ceramide 1, ceramide 2, ceramide 3, ceramide 4, ceramide 5, ceramide 6, hydroxypropyl bispalmitamide MEA, and hydroxypropyl bislauramide MEA, and combinations thereof.

Examples of peptides that interact with protein structures of the dermal-epidermal junction include palmitoyl dipeptide-5 diaminobutyloyl hydroxythreonine and palmitoyl dipeptide-6 diaminohydroxybutyrate.

Examples of vitamins include, but are not limited to, vitamins A, D, E, K, and combinations thereof. Vitamin analogues are also contemplated; for example the vitamin D analogues calcipotriene or calcipotriol.

In certain embodiments, the vitamin may be present as tetrahexyldecyl ascorbate. This compound exhibits anti-oxidant activity, inhibiting lipid peroxidation.

Examples of sunscreens include, but are not limited to, p-aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, zinc oxide, 4-methylbenzylidene camphor, methylene bis-benzotriazolyl tetramethylbutylphenol, bis-ethylhexyloxyphenol methoxyphenyl triazine, terephthalylidene dicamphor sulfonic acid, drometrizole trisiloxane, disodium phenyl dibenzimidazole tetrasulfonate, diethylamino hydroxybenzoyl hexyl benzoate, octyl triazone, diethylhexyl butamido triazone, polysilicone-15, and combinations thereof.

Suitable fragrances and colors may be used in the formulations of the present invention. Examples of fragrances and colors suitable for use in topical products are known in the art.

Suitable immunomodulators include, but are not limited to, tetrachlorodecaoxide, deoxycholic acid, tacrolimus, pimecrolimus, imiquimod, and beta-glucan.

Often one constituent of a composition may accomplish several functions. In one embodiment, the present invention relates to constituents that may act as a lubricant, an emollient, or a skin-penetrating agent. In one embodiment, the multi-functional constituent is socetyl stearate, isopropyl isostearate, isopropyl palmitate, or isopropyl myristate.

### Exemplary Compositions or Formulations of the Invention

In certain embodiments, the invention relates to a composition or a formulation comprising a therapeutically effective amount of a tyrosine kinase inhibitor, and a dermatologically acceptable carrier or excipient.

In certain embodiments, the invention relates to any one of the aforementioned compositions or formulations, wherein the tyrosine kinase inhibitor is effective against BCR-ABL tyrosine kinase, c-Abl tyrosine kinase, α-PDGFR, β-PDGFR, or KIT receptor kinase, or inhibits TGF-β.

In certain embodiments, the invention relates to any one of the aforementioned compositions or formulations, wherein the tyrosine kinase inhibitor is imatinib or nilotinib.

In certain embodiments, the invention relates to any one of the aforementioned compositions or formulations, wherein the tyrosine kinase inhibitor is imatinib.

In certain embodiments, the invention relates to any one of the aforementioned compositions or formulations, wherein the tyrosine kinase inhibitor is AG 18, DMPQ, PD 166285, PPY A, SU 16f, SU 5416, SU 6668, or sunitinib.

In certain embodiments, the invention relates to any one of the aforementioned compositions or formulations, wherein the tyrosine kinase inhibitor is dissolved in the carrier or excipient.

In certain embodiments, the invention relates to any one of the aforementioned compositions or formulations, wherein the tyrosine kinase inhibitor is dispersed in the carrier or excipient.

In certain embodiments, the invention relates to any one of the aforementioned compositions or formulations, wherein the carrier or excipient is a gel.

In certain embodiments, the invention relates to any one of the aforementioned compositions or formulations, wherein the carrier or excipient is an anhydrous gel.

In certain embodiments, the invention relates to any one of the aforementioned compositions or formulations, wherein the carrier does not comprise a substantial quantity of water.

In certain embodiments, the invention relates to any one of the aforementioned compositions or formulations, wherein the carrier or excipient is a hydrophilic anhydrous gel.

### Exemplary Properties of Compositions or Formulations of the Invention

In certain embodiments, the invention relates to any one of the aforementioned compositions or formulations, wherein the composition or formulation is a cream, a lotion, a solution, a gel, or an ointment.

In certain embodiments, the invention relates to any one of the aforementioned compositions or formulations, wherein the composition or formulation is a spray. In certain embodiments, the invention relates to any one of the aforementioned formulations, wherein the formulation is an aerosol spray.

In certain embodiments, the invention relates to any one of the aforementioned formulations that, upon expulsion from an aerosol container, forms a foam.

In certain embodiments, the invention relates to any one of the aforementioned compositions or formulations that, upon application to the skin of an affected subject, is non-irritating.

In certain embodiments, the invention relates to any one of the aforementioned compositions or formulations that, upon application to the skin of an affected subject, is well-tolerated.

In certain embodiments, the invention relates to any one of the aforementioned compositions or formulations that, upon application to the skin of an affected subject, reduces inflammation.

In certain embodiments, the invention relates to any one of the aforementioned compositions or formulations that, upon application to the skin of an affected subject, is non-cytotoxic.

In certain embodiments, the invention relates to any one of the aforementioned compositions or formulations that, upon application to the skin of an affected subject, does not produce edema or erythema.

In certain embodiments, the invention relates to any one of the aforementioned compositions or formulations, wherein an assay for the quantity of the tyrosine kinase inhibitor shows greater than about 70% of the initial quantity of tyrosine kinase inhibitor in the composition or formulation after storing the composition or formulation for about 1 week, about 2 weeks, about 3 weeks, about 4 weeks, about 6 weeks, about 8 weeks, or about 12 weeks.

In certain embodiments, the invention relates to any one of the aforementioned compositions or formulations, wherein the assay shows greater than about 80%, greater than about 90%, or greater than about 95% of the initial quantity of tyrosine kinase inhibitor in the composition or formulation after storing the composition or formulation for about 1 week, about 2 weeks, about 3 weeks, about 4 weeks, about 6 weeks, about 8 weeks, or about 12 weeks.

In certain embodiments, the invention relates to any one of the aforementioned compositions or formulations that, upon application to the skin of an affected subject, reduces the expression of a biomarker selected from the group consisting of those outlined in the following table.

AXIN2
Acta2
Adam12
Angpt2
Arg1
CCL2
CCL4
CCL5
CD14
CD163
CXCL10
CXCL2
CXCL5
CXCL9
Chi3l1
Chi3l3
Col1a1
Cspg4
Edn1
Fmod
GREM2
IL1b
IL33
IL6
IRF5
IRF7
Icam1
Il13ra1
Itgam
LOX
MX2
Mcam
Mfge8
Mmp12
Mmp13
Ngfr
Nos2
OAS1
Retnla
Rgs5
SPP1
Serpine1
Sfrp2
TNF
Thbs1
Timp1
Vwf
WISP1
Actb
Api5
Eef1a1
Ndufc2
Rnf44
Rpl36al
Rpl9
Rps7
Rwdd1
Sf3b2
Tuba1b

In certain embodiments, the invention relates to any one of the aforementioned compositions or formulations that, upon application to the skin of an affected subject, reduces the expression of a biomarker selected from the group consisting of: Acta2, Adam12, Angpt2, Col1a1, Fmod, LOX, SPP1, Serpine1, Sfrp2, Thbs1, and WISP1.

In certain embodiments, the invention relates to any one of the aforementioned compositions or formulations that, upon application to the skin of the affected subject, reduces the expression of the biomarker in a first sample of skin, wherein the composition or formulation was applied to the first sample of skin.

In certain embodiments, the invention relates to any one of the aforementioned compositions or formulations that, upon application to the skin of the affected subject, reduces the expression of the biomarker in a second sample of skin, wherein the composition or formulation was not applied to the second sample of skin.

In certain embodiments, the invention relates to any one of the aforementioned compositions or formulations that, upon application to the skin of an affected subject, reduces the expression of a biomarker, as compared with the expression of the biomarker in untreated skin.

### Exemplary Compositions or Formulations of the Invention for Particular Uses

In certain embodiments, the invention relates to any one of the aforementioned compositions or formulations for use in the topical treatment of scleroderma.

In certain embodiments, the invention relates to any one of the aforementioned compositions or formulations for use in the treatment of scleroderma, wherein the composition is formulated for topical application once daily, twice daily, or three times daily.

### Exemplary Methods of Use

In certain embodiments, the invention relates to a method of treating scleroderma, comprising the step of applying topically to an affected area of skin of a subject in need thereof a therapeutically-effective amount of any one of the aforementioned compositions or formulations.

In one embodiment, the present invention relates to any one of the above-mentioned methods, wherein the subject is a human.

In one embodiment, the present invention relates to any one of the above-mentioned methods, wherein the composition is applied once daily.

In one embodiment, the present invention relates to any one of the above-mentioned methods, wherein the composition is applied twice daily.

In one embodiment, the present invention relates to any one of the above-mentioned methods, wherein the composition is applied three times daily.

In one embodiment, the present invention relates to any one of the above-mentioned methods, wherein the composition is applied more than three times daily.

In one embodiment, the present invention relates to any one of the above-mentioned methods, wherein the scleroderma is localized.

In one embodiment, the present invention relates to any one of the above-mentioned methods, wherein the scleroderma is systemic.

In one embodiment, the present invention relates to any one of the above-mentioned methods, wherein the scleroderma is associated with inflammation or sclerosis.

In one embodiment, the present invention relates to any one of the above-mentioned methods, wherein the scleroderma is associated with atrophy.

In one embodiment, the present invention relates to any one of the above-mentioned methods, wherein the scleroderma is progressive.

In one embodiment, the present invention relates to any one of the above-mentioned methods, wherein the scleroderma is in remission.

### EXEMPLIFICATION

The following examples are provided to illustrate the invention. It will be understood, however, that the specific details given in each example have been selected for purpose of illustration and are not to be construed as limiting the scope of the invention. Generally, the experiments were conducted under similar conditions unless noted.

### Example 1

### Imatinib Mesylate Formulation for Topical Applications

### Background

The active ingredient, imatinib mesylate (IM), is well-characterized and is known to be safe and effective as a systemic chemotherapeutic agent. IM is approved by the US Food and Drug Administration (FDA), and marketed under the trade name Gleevec®. IM may be used to treat chronic myelogenous leukemia (CML), gastrointestinal stromal tumors (GISTs) and other malignancies. IM acts as a selective inhibitor of BCR-ABL tyrosine kinase, with additional activities against α-PDGFR, β-PDGFR, and KIT receptor kinase. To our knowledge, to date no studies have examined the pharmacodynamics effects of topically administered IM. One object of the invention is to develop a topical formulation in treating the connective tissue disease systemic sclerosis (SSc) or scleroderma.

### Summary

It was found that the targeted IM concentration (20%) was achieved in 3 out of 20+ solvents (including aqueous buffers), used either alone or in mixtures. IM's solubility aqueous solution (for example, Dulbecco's Phosphate Buffered Saline) depends on its concentration, pH, and the (solution) storage conditions. It was possible to prepare a 0.01% or 169.6 μM solution (pH 6.4) which was stable (both chemically and physically) at room temperature for 4 weeks and expected to be stable for a longer time. At pH 7.4 and room temperature the saturation concentration was around 253.0 μM.

Several hydroxypropyl cellulose-based gel prototypes (Lot 1318-36, 2% IM gel, initial pH 6.7; Lot 1318-41, 0.4%) containing benzyl alcohol and ethanol (90%) were prepared and used in animal model studies. A solution of IM in Dulbecco's Phosphate Buffered Saline (NB1318-25, 0.01% or 162 μm IM in 1× buffer, pH=6.5) was also used for initial screening/tissue culture studies.

A stability study (long-term and accelerated) was conducted in which the prototype 2% imatinib mesylate gel formulation was packaged in 2 mL amber glass scintillation vials. The data showed that gels were suitably stable after 1 month under a range of storage conditions.

### Chemistry

Name: Imatinib Mesylate

The active substance is the mesylate salt of the phenylaminopyridine derivative imatinib, 4[(4-methylpiperazin-1-yl)methyl]-N-[4-methyl-3-[(4-pyridin-3-ylpyrimidin-2-yl)amino]phenyl]benzamide methanesulfonic acid.

Chemical Name: methanesulfonic acid; 4-[(4-methylpiperazin-1-yl)methyl]-N-[4-methyl-3-[(4-pyridin-3-ylpyrimidin-2-yl)amino]phenyl]benzamide

CAS: 220127-57-1

UV/Vis: 238, 271 nm

Molecular weight: 589.7084 g/mol

Molecular Formula: C₂₉H₃₁N₇O.CH₄SO₃

Descriptions: White to yellow or brown tinged crystalline powder

Partition Co-efficient (Log P): 1.27

Aqueous Solubility (reported in the literature): Soluble in aqueous buffers≦pH 5.5, very slightly soluble in neutral to alkaline aqueous buffers.

Non-aqueous Solubility (reported in the literature): Freely soluble to very soluble in dimethyl sulfoxide, methanol, ethanol, and dimethyl formamide; insoluble in n-octanol, acetone, and acetonitrile.

Solid-State Forms: Crystalline with two known polymorphic forms (A and B)

### Solubility Study

Solubility of IM was tested in aqueous and organic solvents as described below:

Solubility in an Aqueous Buffer:

Dulbecco's Phosphate Buffered Saline (without Calcium, without Magnesium, source: Sigma Aldridge, Product #59331C-1000ML) was used. The stock buffer (10×) solution was diluted (1:9 dilution) with purified water (final concentration: 1×, final pH=7.24) and used for the solubility study. The solid drug substance (IM) was purchased from Eton biosciences, Inc. (Product SKU#1100170053).

Solubility experiments were performed at two different pHs. To achieve pH 6.5, the pH of the buffer (1×) was adjusted by using 2% Phosphoric acid. Different amounts of IM were added to the buffer solutions to prepare different stock solutions of the drug. The resulting solutions were packaged in clear glass scintillation vials and stored at room temperature, refrigeration (2-8° C.), and frozen (−20° C.).

Observations:

The drug substance was readily soluble over the range of studied concentrations, and resulted in pale yellow solutions free of visible particulates. After 1 week, the 0.1% and 0.05% compositions had precipitated at all storage temperatures. The 0.01% samples showed no sign of precipitation at any temperature. Assay and solubility data were collected on the selected compositions (Table 1).

TABLE 1
Imatinib Mesylate: Assay and Solubility Data.

| | | Reported drug | |
|---|---|---|---|
| | | content in the | |
| Sample | | solution | |
| lot # and | Storage duration/ | (Avg. | Saturation |
| Descriptions | conditions | % Recovery) | Concentration |
| NB1318-09 | 2 weeks at room | 14.95% | 0.015 wt. % or |
| (Drug added: | temperature | | 253.51 μM |
| 1.0 mg/mL, | 2 weeks, | 3.65% | 0.004 wt. % or |
| 0.10 wt. %) in | refrigerated | | 6.189 μM |
| DPBS Buffer | condition | | |
| (1X, pH 7.24) | 2 weeks, | 56.8% | 0.057% or |
| | refrigerated | | 96.3187 μM |
| | condition | | |
| NB1318-14 | 4 weeks at room | 98.85% | >0.010 wt. % or |
| (Drug added: | temperature | | 167.63 μM (saturation |
| 0.1 mg/mL, | | | was not achieved) |
| 0.01 wt. %) in | | | |
| DPBS Buffer | | | |
| (1X, pH 6.5) | | | |

Solubility in Organic Solvents:

The solubility of imatinib was tested in more than 20 organic solvents. Solubility was tested at room temperature by continuously stirring nominally 0.01% of IM in a pure solvent of interest in a glass scintillation vial. After examining each vial for visual clarity, additional drug substance was added when the solution was free of particulates, and the saturation concentration was noted when particulates were observed. This process was repeated until the saturation was reached for each vial. From the solvents studied, three were identified for further study based on physiochemical and safety properties (Table 2).

TABLE 2
Lead solvents for solubilizing Imatinib Mesylate

| Solvent | IM solubility (5%) |
|---|---|
| propylene glycol | <2.2% |
| benzyl alcohol | <1.6% |
| ethyl alcohol | >20% |

### Prototype Formulations

Taking advantage of the miscibility of benzyl alcohol, and its use at high levels in prescription formulations, gel prototype formulations containing ethanol and benzyl alcohol were developed (Table 3) and packaged in 2 mL amber glass scintillation vials or 100 mL glass jars.

TABLE 3
Prototype gel formulations for the proof-of-concepts study.

| | Wt % | | |
|---|---|---|---|
| | | | NB1318-35 |
| Ingredients | NB1318-36 | NB1318-41 | (Vehicle) |
| 90% Ethyl | 58.8 | 60.4 | 60.8 |
| alcohol | | | |
| Benzyl alcohol | 37.2 | 37.2 | 37.2 |
| USP | | | |
| HPC HFX | 2 | 2 | 2 |
| Imatinib | 2 | 0.4 | — |
| Mesylate | | | |
| Total | 100 | 100 | 100 |

A manufacturing procedure for the gel prototypes was developed as described below:

Gel Manufacturing Procedure:
1. Record the tare weights.
2. Weigh 90% ethanol and benzyl alcohol in a cleaned SS beaker.
3. Weigh hydroxypropyl cellulose HFX in a separate container and keep aside.
4. Weigh and add IM to step I, mix (using stirrer while vessel is covered) until IM (API) is completely dissolved.
5. Transfer the manufacturing vessel from step 3 to Silversion homogenizer (equipped with fine screen) and start mixing at a speed around dial #3 while keeping the stator head just below the surface of the solution.
6. While mixing (step 3), add hydroxypropyl cellulose HFX from step 2 to step 4. Adjust the stirring speed, as necessary, as the viscosity increases. Continue mixing for about 6 min while rotating the manufacturing vessel manually to achieve a homogenous dispersion/continuity of the gel.
7. Remove the vessel from the homogenizer and collect residual product from the mixture and vessel using a spatula.
8. Record the final weight of the product.

### Stability Study:

A stability study was initiated using the 2% IM gel (lot NB1318-36). Table 4 shows the data. Results showed that IM is physically and chemically stable at all studied conditions.

TABLE 4
Stability study on 2% Imatinib Mesylate gel (Lot NB1318-36)
Table 4A: Chemical stability

| | % Label claim | | |
|---|---|---|---|
| Conditions | T = 0 | 1 month | 2 month |
| 40° C. ± 2° C./75% RH ± 5% RH | 102.9 | 102.8 | 102.7 |

Table 4B: Physical stability

| | T = 0 | | T = 1 month | |
|---|---|---|---|---|
| Conditions | Appearance | pH | Appearance | pH |
| 40° C. ± 2° C./75% RH ± 5% RH | Light | 6.7 | Light | 6.7 |
| | yellow gel | | Yellow gel | |

### Membrane Permeation Study Using Franz Cell

Franz cell studies were performed using 2% Imatinib Mesylate gel (Lot NB1318-36) to study the release profile of the drug through different synthetic membranes. Membranes used were:

1) Tuffryn® membrane, which is composed of hydrophilic polysulfone, has a diameter of 25 mm, and a pore size of 0.45 μm;

2) Strat M® membrane, which is constructed of two layers of polyethersulfone on top of one layer of polyolefin. Total membrane thickness is approximately 300 μm, and the diameter is 25 mm (4.9 cm²).

Experimental:

Detailed experimental parameters are shown in Table 5. Briefly, between 1 g and 1.5 g of the gel was loaded into the donor compartment of the Franz diffusion cell, and the cells were covered with Parafilm® to prevent evaporation of the ethanol from the gel. Samples were taken at time points of 30, 60, 120, 240 and 360 minutes. The media used in the receptor compartment of the Franz cell was 5 mM Potassium Phosphate dibasic buffer:Ethanol (70:30). The temperature of the water bath was maintained at 32.5° C. For each of the membranes, two replicates were done for each gel formation.

TABLE 5
Experimental parameters for the Membrane permeation study
and related HPLC analysis.
Membrane Permeation study

| | |
|---|---|
| Franz cell | Franz Cell Diffusion System # 912-SCT-12S |
| Equipment | from Logan instrument, NJ |
| Membrane | 1) Tuffryn membrane Material: Hydrophilic |
| | polysulfone, 25 mm diameter, Pore size = 0.45 um |
| | 2) Strat M membrane, 25 mm (4.9 cm²) diameter |
| Media | 70:30 (5 mM Potassium Phosphate buffer pH |
| | 4.8:Ethanol) |
| Temperature of | 32.5° C. |
| water bath | |
| Sample time points | 30, 60, 120, 240, and 360 minutes |
| Cells covered with: | Parafilm |
| Amount in each cell | 1-1.5 gm |

HPLC

| | |
|---|---|
| Instrument | Liquid Chromatograph equipped with a UV |
| | Detector/Make: Agilent, Model: 1100 series |
| Buffer | 5 mM Potassium Phosphate buffer pH 4.8 |
| Column | Phenomenex Kinetix 2.6 μm C18 4.6 × 50 mm |
| | Part#: 00B-4462-E0 |
| Column Temperature | 30° C. |
| Run time | 5 mins |
| Mobile phase | Mobile phase: 5 mM Phosphate Buffer pH 4.8 (60%), |
| composition | methanol (25%), acetonitrile (15%) |
| | Elution: Isocratic |
| Flow Rate | 1.0 ± 0.2 mL/min |
| Detection | UV at 264 nm |
| Injection Volume | 20 μL |
| Retention Times | Imatinib: 2.4 ± 1.0 minutes |

Results:

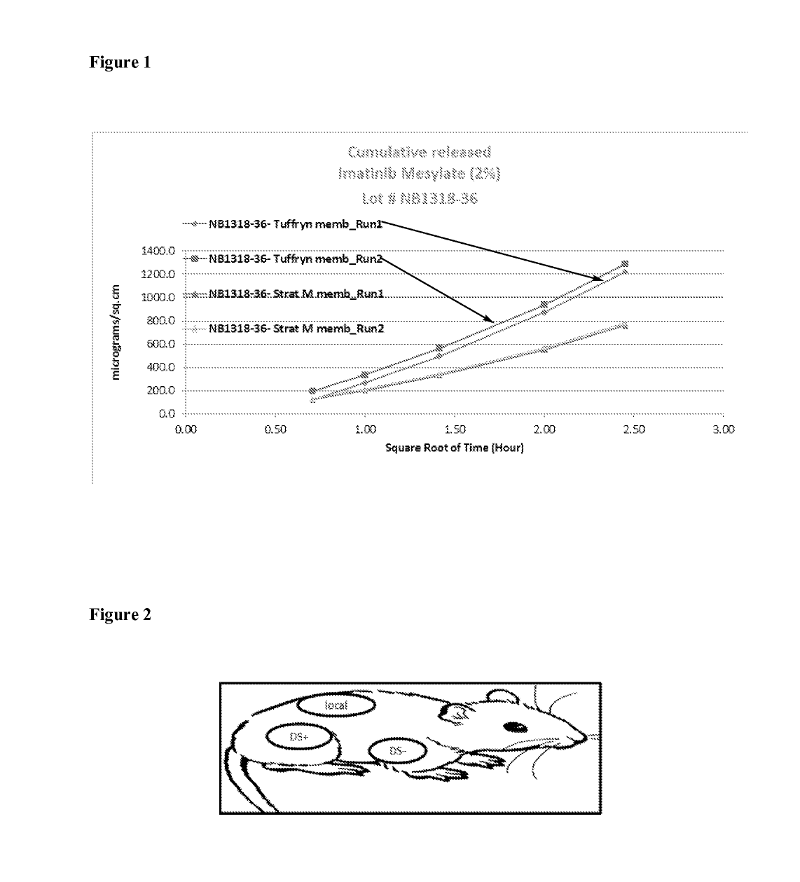

Table 6 shows the extent of membrane permeation, and FIG. 1 shows the permeation time profile for Imatinib Mesylate from the gel formulation. Release of Imatinib Mesylate from the gel formulation was substantially different for the two membranes.

TABLE 6
Membrane permeation data
(Formulation: 2% Imatinib gel, Lot NB1318-36)

| | | Tuffryn | | Strat M | |
|---|---|---|---|---|---|
| Sq. root of | | RUN 1 | RUN 2 | RUN 1 | RUN 2 |
| time | Time (hr) | Avg. Release (μg/cm²) | | | |
| 0.71 | 0.5 | 122.2 | 197.7 | 126.2 | 130.3 |
| 1.00 | 1 | 268.1 | 338.7 | 202.7 | 211.9 |
| 1.41 | 2 | 494.9 | 565.2 | 334.7 | 346.4 |
| 2.00 | 4 | 873.4 | 939.7 | 553.6 | 570.4 |
| 2.45 | 6 | 1218.9 | 1287.6 | 760.4 | 779.2 |

Conclusions:

Results from pre-formulation, prototype formulation, and in vitro performance studies indicate that the selected gel formulation is suitable for topically delivering imatinib mesylate.

### Example 2

### Animal Studies

C57BL/6 mice were obtained from The Jackson Laboratory (Bar Harbor, Me.). Osmotic pumps (Alzet, model 2001) designed to deliver 1 μL/h were loaded with TGF-β (1.25 μg) in phosphate buffered saline (PBS) supplemented with 0.1 mg/mL bovine serum albumin (BSA). Pumps were implanted subcutaneously in 8-week-old mice, who were subsequently treated with imatinib or placebo cream. After 7, mice were sacrificed, and the skin (˜1 cm²) surrounding the pump outlet was homogenized in TRIzol (Invitrogen) for preparation of RNA, or fixed in formalin. Skin from mice treated with PBS and TGF-β was analyzed using nanostring technology. A set of 50 genes including inflammatory genes, macrophages markers, TGF-β-regulated genes, and others were analyzed. 100 ng of RNA per sample was used and gene expression was normalized to the expression of 8 housekeeping-genes.

See, e.g., Christmann, R. B., et al. Arthritis Rheum. 2013 May; 65(5):1335-46 (teaching that thymic stromal lymphopoietin is up-regulated in the skin of patients with systemic sclerosis and induces profibrotic genes and intracellular signaling that overlap with those induced by interleukin-13 and transforming growth factor β).

### Mice:

C57B1/6j 20 g

### Group of 4:

| | | |
|---|---|---|
| n = 1 | PBS | pump |
| n = 1 | TGFb | pump |
| n = 2 | PBS | pump + imatinib cream |
| n = 2 | TGFb | pump + imatinib cream |

### Dose:

TGFb 2.5 μg/mL in 7 day Alzet pump.

imatinib cream topical application 7 days (2 times per day)

### Sacrificed and Collection Tissue (See FIG. 2):

(1) LS=local skin

(2) +DS=Near local skin

(3) −DS=Far from local skin

*half skin sample for RNA study

*half skin sample for Histology study

TABLE 7
Biomarkers related to Scleroderma
AXIN2
Acta2
Adam12
Angpt2
Arg1
CCL2
CCL4
CCL5
CD14
CD163
CXCL10
CXCL2
CXCL5
CXCL9
Chi3l1
Chi3l3
Col1a1
Cspg4
Edn1
Fmod
GREM2
IL1b
IL33
IL6
IRF5
IRF7
Icam1
Il13ra1
Itgam
LOX
MX2
Mcam
Mfge8
Mmp12
Mmp13
Ngfr
Nos2
OAS1
Retnla
Rgs5
SPP1
Serpine1
Sfrp2
TNF
Thbs1
Timp1
Vwf
WISP1
Actb
Api5
Eef1a1
Ndufc2
Rnf44
Rpl36al
Rpl9
Rps7
Rwdd1
Sf3b2
Tuba1b

TABLE 8
Results of mouse studies

| | | | |
|---|---|---|---|
| Gene Name | Group Name | DS+ | LS |

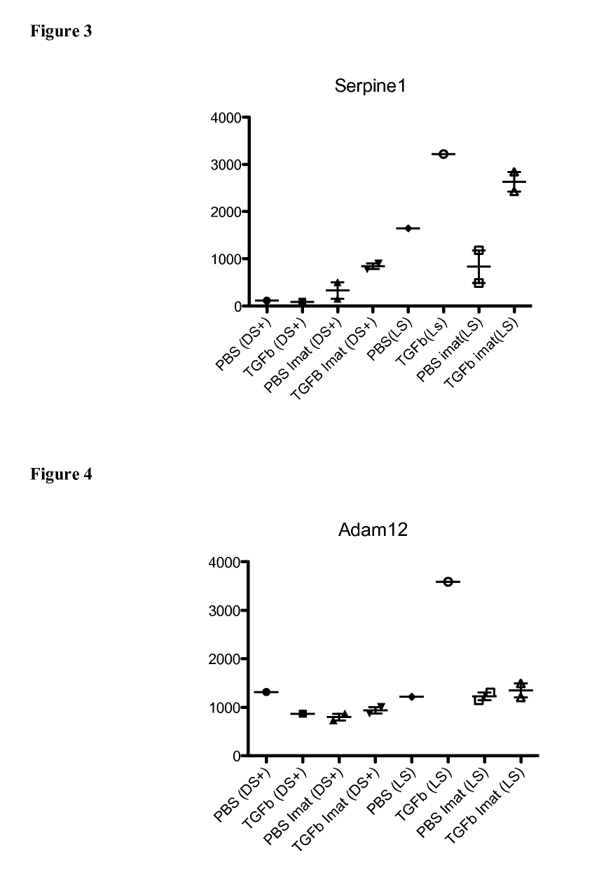

FIG. 3

| | | | |
|---|---|---|---|
| serpine1 | TGFb/PBS | (−) | (+) |
| | TGFb/TGFbimatinib | (−) | (+) |

FIG. 4

| | | | |
|---|---|---|---|
| Adam12 | TGFb/PBS | (−) | (+) |
| | TGFb/TGFbimatinib | (−) | (+) |

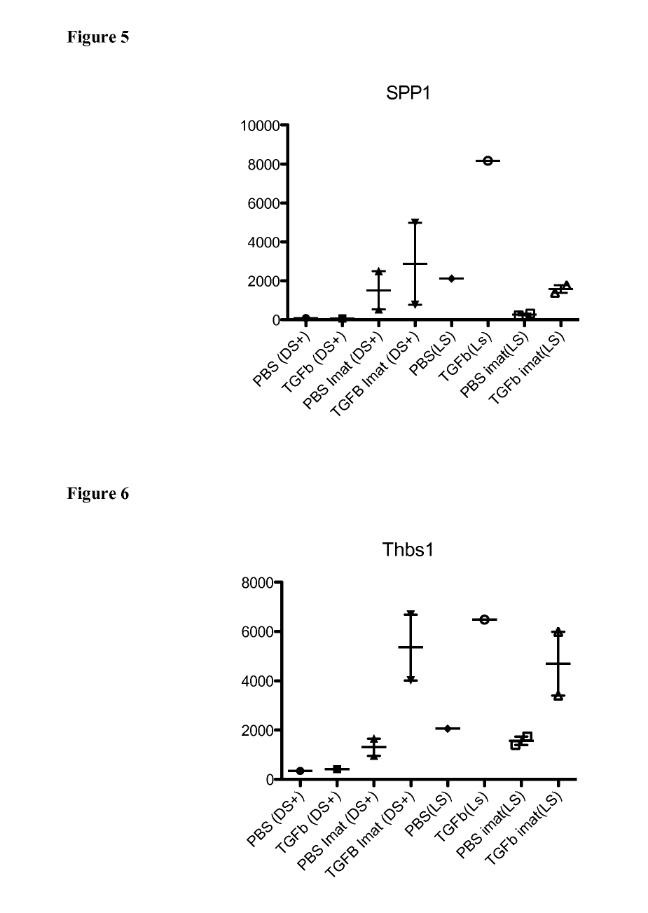

FIG. 5

| | | | |
|---|---|---|---|
| spp1 | TGFb/PBS | (−) | (+) |
| | TGFb/TGFbimatinib | (−) | (+) |

FIG. 6

| | | | |
|---|---|---|---|
| Thbs1 | TGFb/PBS | (−) | (+) |
| | TGFb/TGFbimatinib | (−) | (+?) |

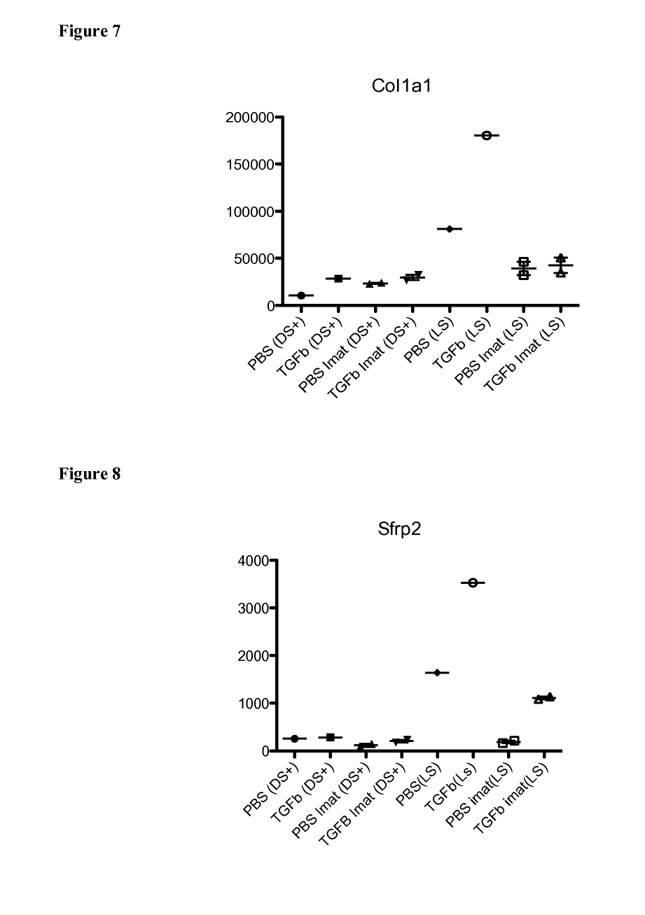

FIG. 7

| | | | |
|---|---|---|---|
| Col1a1 | TGFb/PBS | (+) | (+) |
| | TGFb/TGFbimatinib | (−) | (+) |

FIG. 8

| | | | |
|---|---|---|---|
| Sfrp2 | TGFb/PBS | (−) | (+) |
| | TGFb/TGFbimatinib | (−) | (+) |

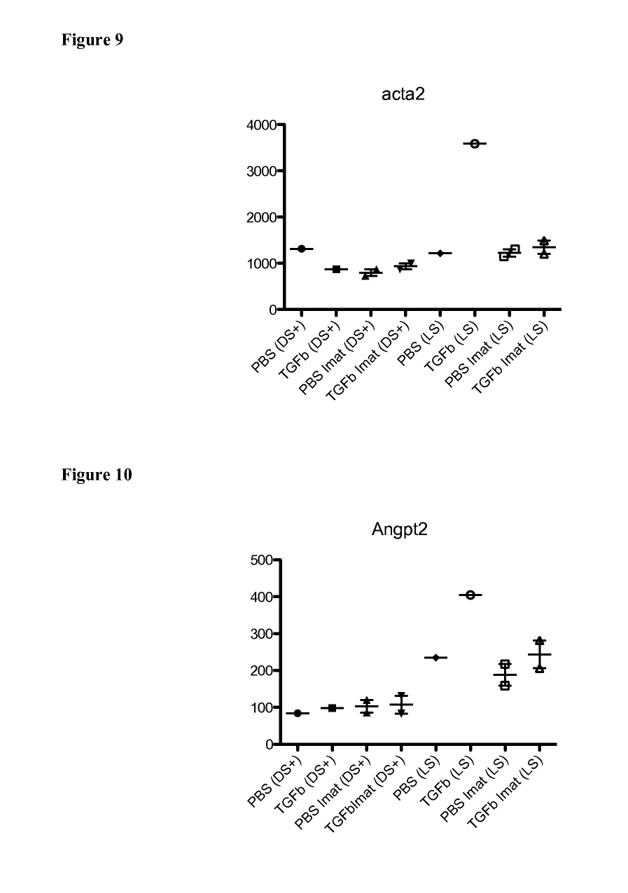

FIG. 9

| | | | |
|---|---|---|---|
| acta2 | TGFb/PBS | (−) | (+) |
| | TGFb/TGFbimatinib | (−) | (+) |

FIG. 10

| | | | |
|---|---|---|---|
| Angpt2 | TGFb/PBS | (−) | (+) |
| | TGFb/TGFbimatinib | (−) | (+) |

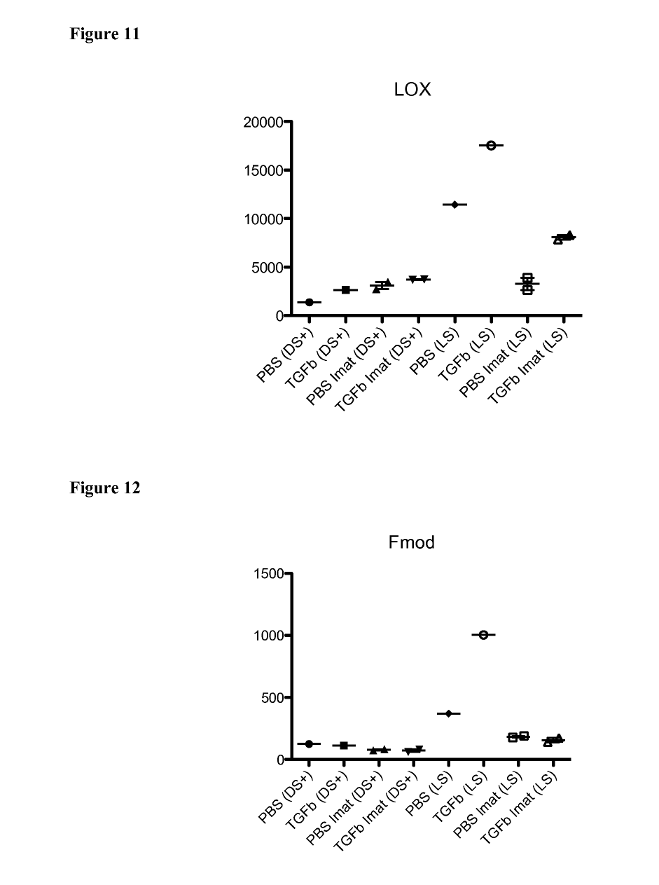

FIG. 11

| | | | |
|---|---|---|---|
| lox | TGFb/PBS | (?+) | (+) |
| | TGFb/TGFbimatinib | (−) | (+) |

FIG. 12

| | | | |
|---|---|---|---|
| Fmod | TGFb/PBS | (−) | (+) |
| | TGFb/TGFbimatinib | (−) | (+) |

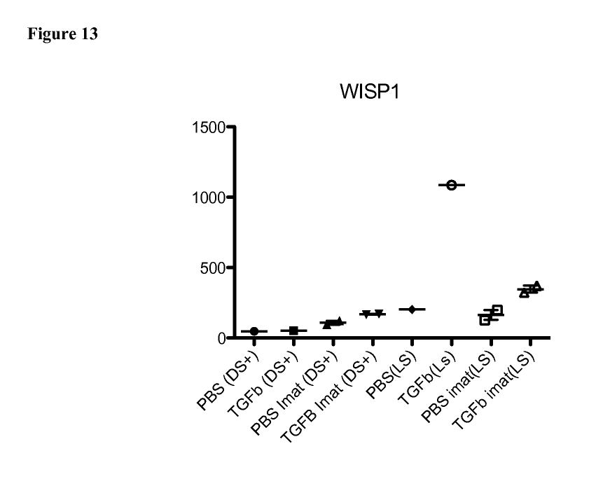

FIG. 13

| | | | |
|---|---|---|---|
| Wisp1 | TGFb/PBS | (−) | (+) |
| | TGFb/TGFbimatinib | (−) | (+) |

### Summary of In Vivo Experiments

TGFβ treatment works and the effect is blocked by imatinib in the local skin (+) and DS+ skin (−).

TGFβ activated genes are upregulated and imatinib inhibits the activation of these genes.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of treating scleroderma, comprising the step of applying topically to an affected area of skin of a subject in need thereof a composition or a formulation comprising a therapeutically-effective amount of a tyrosine kinase inhibitor; and a dermatologically acceptable carrier or excipient.

2. The method of claim 1, wherein the tyrosine kinase inhibitor is effective against BCR-ABL tyrosine kinase, c-Abl tyrosine kinase, α-PDGFR, β-PDGFR, or KIT receptor kinase, or inhibits TGF-β.

3. The method of claim 1, wherein the tyrosine kinase inhibitor is imatinib or nilotinib.

4. The method of claim 1, wherein the tyrosine kinase inhibitor is imatinib.

5. The method of claim 1, wherein the tyrosine kinase inhibitor is AG 18, DMPQ, PD 166285, PPY A, SU 16f, SU 5416, SU 6668, or sunitinib.

6. The method of any one of claims 1-5, wherein the tyrosine kinase inhibitor is dissolved in the carrier or excipient.

7. The method of any one of claims 1-6, wherein the composition or formulation is a cream, a lotion, a solution, a gel, or an ointment.

8. The method of any one of claims 1-7, wherein the carrier or excipient is a gel.

9. The method of claim 8, wherein the carrier or excipient is an anhydrous gel.

10. The method of any one of claims 1-6, wherein the composition or formulation is a spray.

11. The method of any one of claims 1-10, wherein the composition or formulation is non-irritating.

12. The method of any one of claims 1-11, wherein the composition or formulation is well-tolerated.

13. The method of any one of claims 1-12, wherein the composition or formulation reduces inflammation.

14. The method of any one of claims 1-13, wherein the composition or formulation is non-cytotoxic.

15. The method of any one of claims 1-14, wherein the composition or formulation does not produce edema or erythema.

16. The method of any one of claims 1-15, wherein the subject is a human.

17. The method of any one of claims 1-16, wherein the composition or formulation is applied once daily.

18. The method of any one of claims 1-16, wherein the composition or formulation is applied twice daily.

19. The method of any one of claims 1-16, wherein the composition or formulation is applied three times daily.

20. The method of any one of claims 1-19, wherein the scleroderma is localized.

21. The method of any one of claims 1-20, wherein the scleroderma is associated with inflammation or sclerosis.

22. The method of any one of claims 1-21, wherein the scleroderma is associated with atrophy.

23. The method of any one of claims 1-22, wherein the scleroderma is progressive.

24. The method of any one of claims 1-22, wherein the scleroderma is in remission.

25. The method of any one of claims 1-24, wherein the composition or formulation reduces the expression of a biomarker selected from the group consisting of:
AXIN2
Acta2
Adam12
Angpt2
Arg1
CCL2
CCL4
CCL5
CD14
CD163
CXCL10
CXCL2
CXCL5
CXCL9
Chi3I1
Chi3I3
CoI1a1
Cspg4
Edn1
Fmod
GREM2
IL1b
IL33
IL6
IRF5
IRF7
Icam1
Il13ra1
Itgam
LOX
MX2
Mcam
Mfge8
Mmp12
Mmp13
Ngfr
Nos2
OAS1
Retnla
Rgs5
SPP1
Serpine1
Sfrp2
TNF
Thbs1
Timp1
Vwf
WISP1
Actb
Api5
Eef1a1
Ndufc2
Rnf44
Rp136a1
Rpl9
Rps7
Rwdd1
Sf3b2
Tuba1b

26. The method of any one of claims 1-24, wherein the composition or formulation reduces the expression of a biomarker selected from the group consisting of: Acta2, Adam12, Angpt2, Col1a1, Fmod, LOX, SPP1, Serpine1, Sfrp2, Thbs1, and WISP1.

27. The method of claim 25 or 26, wherein the expression of the biomarker is reduced in a first sample of skin, wherein the composition or formulation was applied to the first sample of skin.

28. The method of any one of claims 25-27, wherein the expression of the biomarker is reduced in a second sample of skin, wherein the composition or formulation was not applied to the second sample of skin.

29. The method of any one of claims 25-28, wherein the expression of the biomarker is reduced as compared to the expression of the biomarker in untreated skin cells.

30. A composition or a formulation, comprising a therapeutically effective amount of a tyrosine kinase inhibitor; and a dermatologically acceptable carrier or excipient.

31. The composition or formulation of claim 30, wherein the tyrosine kinase inhibitor is effective against BCR-ABL tyrosine kinase, c-Abl tyrosine kinase, α-PDGFR, β-PDGFR, or KIT receptor kinase, or inhibits TGF-β.

32. The composition or formulation of claim 30, wherein the tyrosine kinase inhibitor is imatinib or nilotinib.

33. The composition or formulation of claim 30, wherein the tyrosine kinase inhibitor is imatinib.

34. The composition or formulation of claim 30, wherein the tyrosine kinase inhibitor is AG 18, DMPQ, PD 166285, PPY A, SU 16f, SU 5416, SU 6668, or sunitinib.

35. The composition or formulation of any one of claims 30-34, wherein the tyrosine kinase inhibitor is dissolved in the carrier.

36. The composition or formulation of any one of claims 30-35, wherein the composition or formulation is a cream, a lotion, a solution, a gel, or an ointment.

37. The composition or formulation of any one of claims 30-36, wherein the carrier or excipient is a gel.

38. The composition or formulation of claim 37, wherein the carrier or excipient is an anhydrous gel.

39. The composition or formulation of any one of claims 30-35, wherein the composition or formulation is a spray.

40. The composition or formulation of any one of claims 30-38, wherein upon expulsion from an aerosol container said composition or formulation forms a foam.

41. The composition or formulation of any one of claims 30-40, wherein an assay for the quantity of tyrosine kinase inhibitor shows greater than about 70% of the initial quantity of tyrosine kinase inhibitor in the composition or formulation after storing the composition or formulation for about 2 weeks.
